(19) United States
(12) Patent Application Publication
(10) Pub. No.: US 2009/0177282 A1
(43) Pub. Date: Jul. 9, 2009

(54) IMPLANTABLE BIOMIMETIC PROSTHETIC BONE

(75) Inventors: Martin N. Bureau, Montreal (CA); Jean-Gabriel Legoux, Repentigny (CA); Johanne Denault, Longueuil (CA)

Correspondence Address: BORDEN LADNER GERVAIS LLP;Anne Kinsman, WORLD EXCHANGE PLAZA, 100 QUEEN STREET SUITE 1100, OTTAWA, ON, K1P 1J9, CA

(73) Assignee: National Research Council of Canada

(21) Appl. No.: 11/813,827

(22) PCT Filed: Jan. 13, 2006
(86) PCT No.: PCT/CA2006/000040

§ 371 Date: Jun. 12, 2008

Related U.S. Application Data
(60) Provisional application No. 60/643,599, filed on Jan. 14, 2005.
(60) Provisional application No. 60/676,299, filed on May 2, 2005.

(51) Int. Cl. A61F 2/28; B29C 70/00; B29C 43/02

(52) U.S. Cl. 623/16.11; 623/23.59; 264/328.16; 264/134

(57) ABSTRACT

Bone tissue at the interface of a bone implant is shielded from stresses found in normal bone because of the higher stiffness or rigidity in the implant versus in bone. The resulting “stress shielding” of the bone by the implant eventually results in resorption of bone at the bone-implant interface and ultimately necessitates replacement of the bone implant. To overcome these problems, an implantable biomimetic prosthetic bone having a porous surface, a fiber-reinforced composite structure, and a polymer-based core is disclosed. The prosthetic bone is a good match for structure, stiffness, viscoelastic properties, specific weight and overall structure as real bone or host tissues adjacent to the prosthetic bone. The prosthetic bone may be formed as a total hip prosthesis.

### CROSS REFERENCE TO RELATED APPLICATIONS

This application claims the benefit of priority of U.S. Provisional Patent Applications No. 60/643,599 filed Jan. 14, 2005, and No. 60/676,299 filed May 2, 2005, each of which is incorporated herein by reference.

### FIELD OF THE INVENTION

The present invention relates generally to implantable prosthetic materials, in particular to biomimetic composite prosthetic materials and prostheses made of such materials.

### BACKGROUND OF THE INVENTION

Metallic prosthetic implants have enjoyed enormous success for replacing bone and for bone fracture fixations and repairs. Among those, the Charnley-type hip replacement implant became an orthopedic success story as the second most frequently performed surgical procedure after the appendix ablation.

The hip joint is a ball-and-socket joint in which the spherical head of the thighbone (femur) moves inside the cup-shaped hollow socket (acetabulum) of the pelvis. To duplicate this action, a total hip replacement implant or total hip prosthesis (THP) has three parts: a stem, which is inserted into the femur, a femoral head (a ball) which replaces the spherical head of the femur, and an acetabular cup which replaces the worn-out or otherwise damaged hip socket, the cup remaining in contact with the head. In some designs, the stem and ball are one piece; other designs are modular, allowing for additional customization in fit.

Presently, stem portions of most hip implants are made of metallic alloys, usually of stainless steel or titanium- or cobalt/chromium-based alloys. However, total hip prostheses (THPs) having a solid metallic stem usually have to be replaced after a certain number of years, with 10-15% of all THPs being replaced after 10-15 years. While this could be acceptable for older, less active patients, this retrieval rate of THP is clearly not appropriate for younger patients, for which considerably longer implantation periods are required. Ideally, the implantation period should exceed the life span of the patient and restore completely the biomechanical function of the hip without pain. Such an extended life time of the implant is also desirable on economic grounds, bearing in mind that the estimated cost of the first implantation is about $10,000.00, whereas that of a revision (second operation) can reach $20,000.00 to $30,000.00, not counting the costs in loss of productivity related to considerably longer convalescence periods in the case of a revision.

The main problem with presently used THPs lies in a phenomenon known as aseptic loosening, which is attributed to a stiffness mismatch between the bone and the implant. One cause of this phenomenon is stress shielding, while but formation of wear debris is also widely reported as a contributor to this problem. Under normal functional loading, bone tissues at the bone-implant interface are submitted to stresses in first approximation proportional to the rigidity ratio between the implant material and the bone. For implants with metallic stems, the elastic modulus of the materials composing the stem is between 140 GPa and 210 GPa, while that of dense bone material is between 5 GPa and 30 GPa. Under a given stress, the strain at the both sides of the THP interface is the same. As a result, the stress in bone tissues at the THP interface is approximately 2-20% of the stress in the THP. This effect, designated as stress shielding, results in the bone surrounding the THP being underloaded with respect to a normal bone. This leads to gradual resorption of the bone at the bone-implant interface, a phenomenon explained by Wolff's law according to which bone is deposited in sites subjected to normally occurring stresses and resorbed from sites where there is little stress. This eventually generates an inflammatory response of the body causing pain to the host and requiring removal of the implant. When replaced, metallic stems of THPs need to be inserted deeper into the femoral bone, which makes it progressively weaker and increases the risk of fracture. In addition to bone weakening, the metal of the stem may suffer corrosion fatigue and can cause adverse tissue reactions.

A number of attempts at improving the performance of the metallic hip implants have been made, mostly by introducing an osteo-conductive porous structure at their surface. Such implants, however, are susceptible to fatigue and debris formation and do not eliminate the problem of stress shielding and the associated bone resorbtion.

In a brief discussion of prior art THPs which follows, acronyms designating plastic materials used as components of various composite structures have the following meanings: PET, polethylene terephthalate; PBT, polybutylene terephthalate; PSU, polysulfone; PES, polyethersulfone; PAS, polyarylsulfone; PPS, polyphenylene sulfide; PC, polycarbonate; PA, polyamide; PAI, polyamide-imide; TPI, thermoplastic polyimide; PAEK, polyaryletherketone; PEEK, polyetheretherketone; PAEN, polyarylethernitrile; PE, polyethylene; PP, polypropylene; and PEK, polyetherketone.

Other than improvements to metallic stems, various polymer-based stems have been proposed, such as stems constituted of a high-modulus internal plastic core covered with a softer bioinert polymer (U.S. Pat. No. 4,662,887). This publication, and all others mentioned herein are incorporated by reference. However, this approach does not address the need for matching by the stem the bone stiffness, density and structure, and does not eliminate the problem of stress shielding.

More complex stems constituted of an internal core covered with a softer biocompatible polymer have been proposed (EP 277,727; U.S. Pat. No. 5,064,439; U.S. Pat. No. 5,192,330; and JP 01015040). The internal core of the stem is composed of a multi-layer laminate of oriented continuous fibre composite (carbon, glass, polyolefins, PEEK, PET) with a biocompatible matrix (PSU, PES, PAS, PPS, PC, aromatic PA, aromatic PAI, TPI, PAEK, PEEK, PAEN, aromatic polyhydroxyether, thermosetting phenolics, and medical grade epoxides), which may be bioresorbable or not. Various orientations of the fibres are proposed. This approach does not address the need for the stem to match bone density and structure. As the bone modulus match is limited to in-plane stiffness components, the moduli normal to the laminate structure cannot be modulated, which does not eliminate completely the stress shielding.

Variations of the latter approach have been proposed (U.S. Pat. No. 5,163,962; EP 649,640; JP 04226649; U.S. Pat. No. 5,522,904). These designs consist of a stem having a higher stiffness in the section closer to the femoral head, in some cases having some layers of reinforcing fibers positioned in a predetermined orientation. While addressing the problem of stress shielding, this approach does not address the need for matching by the stem the bone density and structure.

Stems constituted of a sheath with an internal core have also been proposed (EP 572,751; U.S. Pat. No. 5,714,105). The sheath is composed of braided continuous fibre thermoplastic composite tows and the core is composed of a thermoplastic fibre reinforced polymer, with fibers preferably oriented longitudinally with respect to the stem. Specific orientations of the braiding are considered. Fibers are either polymeric in nature (polyacrylates, PAEK, PC, PES, PE and PP) or made of carbon or aramid. A metallic grid to improve bone fixation at the surface of the stem is included. The stem is prepared by thermoplastic consolidation, using compaction and heating in a closed mold. The molding process is very complex and expensive, because the process requires very high compaction pressures. Controlling the orientation of the composite sheath is problematic. This approach does not address the need for the stem to match the bone stiffness, density and structure, and the problem of eliminating the stress shielding is not explicitly addressed.

Other stems constituted of a sheath and an internal core have been proposed (U.S. Pat. No. 4,902,297; EP 642,774). The sheath is composed of braided fibers, the nature of which is not disclosed. The core is composed of a thermoplastic discontinuous fibre reinforced polymer, preferably oriented longitudinally with respect to the stem. A layer of a polymer is over-molded onto the stem to define its topography, followed by a consolidation-like process. Neither the specific nature of the constituents of the stem nor the stem properties are disclosed. The design is limited to a stem of constant section and uniform structure. Another embodiment includes a transverse orientation of fibers in the sheath. Means for attaching to the stem a femoral head and the possibility of integrating with the stem a porous surface for bone fixation are also disclosed. However, this design does not address the need for the stem to match the bone stiffness, density and structure. While the stiffness of the stem can be varied in this design, the range of such adjustments is fairly limited due to its longitudinally fibre reinforced polymer core and there is no explicit mention of providing a solution to the problem of stress shielding.

Stems constituted of two concentric cylindrical fibre reinforced sheaths, an internal sheath with a longitudinal fibre orientation and an external sheath with wound fibers, have also been proposed (U.S. Pat. No. 5,141,521; U.S. Pat. No. 5,397,358). An internal core is injected into the concentric sheaths and either pressure-consolidated or chemically cured. The core does not contribute to the mechanical strength of the stem. Another version of this design includes filament winding or braiding in the external sheath and different orientations of the fibers in the external sheath are proposed. The internal sheath is essentially constituted of longitudinally orientated continuous fibers. The nature of the sheath materials is not disclosed, only the proportion of fibres and matrix (70% wt. and 30% wt., respectively) and an irregular surface profile is considered to improve bone fixation. As for earlier discussed designs, this design does not address the need for the stem to match the bone stiffness, density and structure. While the stiffness of the stem can be varied in this design, the range of such adjustments is fairly limited, to values above the range of femoral bone moduli, due to the longitudinally fibre reinforced polymer core. Again, there is no explicit mention of solving the problem of stress shielding.

Other stems with an oriented, fibre-reinforced sheath and a rigid core have been proposed (U.S. Pat. No. 5,181,930; WO 93/19699; U.S. Pat. No. 5,443,513). The internal core of the stem is constituted of a continuous carbon fibre thermoplastic PEEK composite oriented essentially parallel to the stem longitudinal direction. The sheath is also composed of a continuous carbon fibre thermoplastic PEEK composite obtained by filament winding. The stiffness of the stem can be adjusted by varying the orientation of the fibers in the sheath and the thickness of the latter, as well as the dimension of the internal core. A modulus of the core material above 69 GPa, a modulus of the sheath materials between 14 and 69 GPa, and a modulus of the thermoplastic matrix used in the composites below 14 GPa are disclosed. Discontinuous carbon fibre reinforced thermoplastic PEEK composite is considered for the surface of the sheath and there is an explicit mention that the external sheath at any point adjacent to the bone has a modulus similar to the latter. While matching the modulus of the bone and the material at surface of the stem is considered, the overall bone modulus cannot be matched using this design, nor can the bone density and structure. While the modulus of the stem surface material can be varied in this design, the range of stiffness that can be obtained from the stem is considerably above the range of femoral bone stiffness due to the longitudinally fibre reinforced polymer core and there is no explicit mention of providing a solution to the stress shielding. A modification of the original stem design comprises a core composed of a short (below 4 mm) carbon fibre reinforced thermoplastic PEEK composite, molded by injection prior to the filament winding of the external sheath. This modified version presents the same limitations as the original one.

Another composite stem composed of up to three layers of continuous fibre reinforcement has been proposed (WO 91/18562; WO 93/13733; U.S. Pat. No. 5,397,365). In this design, the fibers are made of carbon, graphite, glass, or aramid, and are filament wounded with specific orientations to obtain a stem stiffness between 6.9 GPa and 110 GPa. The matrix in the composite is constituted of PSU, PEEK, PEK, thermoplastic polyimide, medical grade epoxide, or polycyanate. Composite tows pre-impregnated with thermoplastic matrix and subsequent consolidation, composite tows pre-impregnated with thermosetting matrix and subsequent chemical curing, or fibre tows with subsequent thermosetting resin injection (RTM) are disclosed. While a large range of mechanical characteristics with a good rigidity/dimension ratio can be obtained for the stem using this design, such a design does not consider the need for the stem material to match the bone modulus, density and structure. The design also does not include an internal core, which considerably increases the risk of buckling of the stem. While admitting that the bone adjacent to the stem is subjected to flexural stresses similar to those experienced when a metallic stem is used, this design does not address the problem of stress shielding.

The last category of stems proposed in the prior art includes fibrous sub-elements of multi-layered fibre reinforced composites (WO 90/12994). The orientations of fibers in each sub-element can be adjusted to obtain pre-determined mechanical characteristics. A thermoplastic matrix (PEEK) or a thermosetting matrix (medical grade epoxide) and fibers of carbon, glass, or polymer-based are used. Each sub-element can include ceramic or metallic components, such as the femoral head element. Assembling of sub-elements can be achieved thermoplastically, either by consolidation (compaction and heating) or by adhesive bonding followed by consolidation. It can also be achieved by thermosetting means, either adhesive bonding and chemical/thermal curing, or by resin injection (RTM) and chemical/thermal curing. This design does not address the need for the stem to match the bone stiffness, density and structure. While the stiffness of the stem can be varied in this design, the range that can be obtained is not explicitly disclosed and there is no explicit mention of providing a solution to the problem of stress shielding. This design also does not include an internal core into the stem structure, which raises considerably the risk of stem buckling.

It is, therefore, desirable to provide a prosthetic bone that is similar in structure to bone, and that has similarities in such physical properties as stiffness/rigidity and strength.

### SUMMARY OF THE INVENTION

It is an object of the present invention to obviate or mitigate at least one disadvantage of previous prosthetic bones, or methods for their formation.

According to an embodiment of the invention, there is provided an implantable biomimetic prosthetic bone formed of a polymer-based core, a fiber-reinforced thermoplastic composite surrounding the core; and a surface comprising an osteo-conductive region. The osteo-conductive region of the surface may comprise a region of porosity, for example with about 10% porosity. Further, the osteo-conductive region of the surface may comprise a region of roughness, for example with meso (100-500 μm), micro (1-50 μm) or nano (<1 μm) roughness. The surface may be bonded to the thermoplastic composite using a tie layer comprising a compatible polymeric matrix and 2-70% filler. Optionally, the surface may comprise an osteo-inductive porous region. The osteo-conductive porous region may comprise a ceramic, or a material that is a combination of ceramic with metal or polymer.

Physical properties of the prosthetic bone may include an elastic modulus of between 5 and 30 Gpa, or a specific weight of from about 0.2 to about 4.0 g/cm³. A range of from 0.4 to 4.0 g/cm³ would also be suitable, and an exemplary range is from 0.4-2.1 g/cm³. The prosthetic bone may have an extra-osseous section and an intra-osseous section, each section having a surface thereon. The said osteo-conductive region being located on the surface of the intra-osseous section, and may be bioresorbable or biodegradable. The surface may also comprises a smooth region, which may be formed of a biocompatible polymer formed of thermoplastic, optionally having a composite structure including short fibers, long fibers, continuous fibers, whiskers, particles, or combinations thereof as filler. The composite structure may include polymer-based oriented fibers; mineral-based fibers; metallic fibers; ceramic fibers; or polymer-based fibers with nanoreinforcement by nanoparticles, nanowhiskers, nanofibers or nanotubes. The surface may comprise hydroxyapatite, TiO₂ or a CaP-containing ceramic, or any of these in combination.

The fiber component of the fiber-reinforced thermoplastic composite may be wrapped in any number of ways, such as braided wound or filament wound around the polymer-based core, and may contain any biocompatible thermoplastic composite or thermoset resin, such as for example CF/PA12.

Further, embodiments of the invention provide a method for forming an implantable biomimetic prosthetic bone comprising the steps of: molding a hollow carbon fiber-reinforced thermoplastic composite in the shape of a bone to be replaced, consolidating the thermoplastic composite with application of heat at a temperature higher than the melting point of the thermoplastic; coating the thermoplastic composite with an osteo-conductive material; and forming a region of roughness or porosity on the surface of the fiber-reinforced thermoplastic composite. The step of coating the thermoplastic composite with an osteo-conductive material may involve applying to the thermoplastic composite a tie layer comprising a compatible polymeric matrix and 2 to 70% filler and subsequently applying the osteo-conductive material. Also the step of forming a region or roughness or porosity may comprise particle sintering, thermal spray coating, or milling. The additional step of forming a smooth region on the surface of the thermoplastic composite layer may be included, which could involve depositing a biocompatible polymer on the thermoplastic composite. For example, depositing the biocompatible polymer may involve overmolding, wrapping, thermal spraying, electrostatic coating, chemical vapour deposition (CVD), electrochemical coating, plasma-spray coating, press-fitting, polymer infiltration, or combinations of these. The fiber-reinforced thermoplastic composite is braided or filament wound. Optionally, a polymer -based core may be injected or inserted into the fiber-reinforced thermoplastic composite.

Advantageously, embodiments of the inventive prosthetic bone can match the bone density (specific weight) and structure of the bone to which the prosthetic bone will become adjacent upon implantation.

As a further advantage, the problem of stress shielding can be in part or wholly overcome with embodiments of the invention that allow for a close stiffness (elastic modulus) match between the materials of the prosthetic bone and the bone to which the prosthetic bone will become adjacent upon implantation.

The presence of an internal core in embodiments of the prosthetic bone of the instant invention advantageously reduces the risk of buckling, as may be found in such prosthetic bone materials that do not include internal cores.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures.

FIG. 1 is a schematic representation of a biomimetic THP according to an embodiment of the invention.

FIG. 2 is a pictorial representation of an exemplary hydroxyapatite (HA) coating on the CF/PA12 (carbon fiber/polyamide 12) composite with a film interlayer according to an embodiment of the invention.

FIG. 3 illustrates a synthetic apatite deposit (upper right), approximately 30 μm in thickness, formed on plasma-sprayed crystalline HA coating from 28-days SBF conditioning at 37° C.

FIG. 4 shows a 3-D finite element model of (a) intact femoral bone and (b) femoral bone with composite prosthesis.

FIG. 5 shows the molding cycle of a hip stem indicating a first rise in temperature to 250° C., maintained for 4 minutes, while the matrix melts and fibers are wetted, then rapid cooling (17° C./min), crystallization and complete solidification.

FIG. 6 illustrates variation of modulus as a function of density depending on the consolidation quality (♦ poor: 175° C., 5 minutes, 50 psi; ▴ medium: 250° C., 4 minutes, 40 psi; [symbol] excellent: 250° C., 4 minutes, 50-90 psi).

FIG. 7 shows two micrographs of stem samples cut and polished in the horizontal plane with: (upper) poor consolidation quality and (lower) high consolidation quality. Resin pockets (dark grey) and large void (black) can be observed in the upper micrograph. Dark spots in carbon fibers correspond to damage created by polishing method.

FIG. 8 shows compression stress-strain curve for composite stems of Example 5.

FIG. 9 illustrates maximum principal stress (MPa) in: (a) intact femoral bone, (b) femoral bone with composite prosthesis and (c) the femoral bone with Ti prosthesis.

FIG. 10 illustrates contact sliding distance (migration) at the proximal bone-implant interface for the CF/PA12 and Ti prostheses of Example 5.

FIG. 11 illustrates the design of the biomimetic hip stem of Example 6.

FIG. 12 shows a schematic of two configurations of fiber architectures used in Example 6.

### DETAILED DESCRIPTION

Generally, the present invention provides biomimetic prosthetic bone implants based on polymer composite technology, which implants possess physical characteristics and overall structure matching most critical physical characteristics and structure of the host tissue adjacent to the implant.

The biomimetic materials according to the present invention can be used for bone implants required or desirable for any reason, such as, but not limited to, for the purpose of a bone repair due its accidental fracture or for an orthopedic correction of an abnormal form or relationship inter-connection of bone structures, or implants to bone structure for attachment of soft tissue such as, but not limited to, ligaments or tendons. In particular, the biomimetic materials of the present invention may be used for repair or replacement of various joints of the human body, such as the shoulder, elbow, wrist, hip, knee, or ankle. Under all circumstances, the implant's biomimetic characteristics, such as its stiffness (isoelasticity), viscoelastic properties, specific weight and overall structure, resemble those of the host tissues adjacent to the implant.

Advantageously, the invention provides an implantable prosthetic bone that possesses physical characteristics matching those of the bone tissue adjacent to the implant, or into which the prosthetic bone becomes implanted.

As a further advantage, the prosthetic bone of the invention can be formulated so as to provide a relatively similar structure to any bone it is intended to replace. For example, the thermoplastic composite corresponds to cortical (dense) bone, while the core corresponds to trabecular (spongy) bone. The outer surface is made to be biocompatible with real bone. In this way, the prosthetic bone is considered to be biomimetic.

According to one embodiment of the invention, a porous osteo-inductive or osteo-conductive surface may be used so as to initiate or perpetuate bone growth. Such a surface may be ceramic and/or metallic in nature. The surface can be biodegradable or bioresorbable in order to promote bone growth at the surface.

For the embodiment in which the prosthetic bone is a stem for implantation as a total hip prosthesis (THP), the stem has biomimetic characteristics resembling those of the femoral bone. In this embodiment, the stem permits adaptation to different commercially available femoral heads, depending on the preference of the orthopedic surgeon. Preferably, such a stem comprises two sections, an extra-osseous section or neck onto which an artificial femoral head can be attached, and a proximal intra-osseous section which fits into the femur. These two sections are composed of continuous or discontinuous, fibre reinforced thermoplastic composite hollow structures, an internal polymer-based core, and different specific types of surface.

The prosthetic bone can be formed in any acceptable shape. For example, it may be cylindrical, frustoconical, or may take on any other shape suitable for insertion in a portion of the bone into which the prosthetic bone is to be implanted, including a shape consistent with the portion of bone to be replaced.

The Composite Layer. The fiber-reinforced thermoset resin or thermoplastic composite may be formed of several concentric layers of a biocompatible polymer composite with specific fiber orientations to obtain the strength and rigidity required for a given application. In the instance where a thermoset resin is used, any resin capable of achieving the biocompatible properties of this layer may be incorporated. In a preferred embodiment, the composite layer comprises a thermoplastic composite. Whether thermoset resin or thermoplastic composite, reinforcing fibers are embedded within the composite layer. Continuous fibers of the composite structure can be polymer-based oriented fibers, or mineral-based fibres, such as, but not limited to, carbon, glass, graphite, or boron fibers. Metallic or ceramic fibers, or polymer-based fibers with nanoreinforcement in the form of, for example (but not limited to) nanoparticles, nanowhiskers, nanofibres or nanotubes can also be used. Carbon fibre polyamide 12 (CF/PA12) composites may be used, such as, for example a composite having 68 wt % long carbon fibers and 32 wt % polyamide 12.

The moduli of the composite layer may range from 5 to 40 GPa and the mechanical strength can range from 100 to 600 MPa.

The composite hollow structure can be braided or filament wound, or be obtained by any other standard composite molding process, and the internal polymer-based core can be either injected or inserted into the composite structure. The structure may be obtained by conventional thermal consolidation procedure (pressure and heat applied over a period of time to ensure complete melting of the polymer matrix and complete wetting of the fibers, followed by controlled cooling until complete solidification) using such processes as would be known to those skilled in the art, for example inflatable bladder molding, compression molding, filament winding, or filament braiding. Alternatively, the composite hollow structure can be built on a core layer.

The Surface. The nature and structure of the surface of the stem can vary along its length in different sections and within a given section. According to one embodiment of the invention, a porous osteo-inductive or osteo-conductive surface may be used that is ceramic or ceramic and metallic in nature. The surface may include a dense region, for example: dense HVOF TiO₂ coating can be made on a Ti surface and we certainly want to be able to include these type of coating. Optionally, the surface can be biodegradable or bioresorbable in order to promote bone growth at the surface. Hydroxyapatite (HA) is one such ceramic surface that is also biodegradable and may be used for application to a prosthetic bone surface, either alone, or in combination with a metal, such as for example Ti.

In the intra-osseous section of the prosthetic bone, which may be inserted adjacent bone tissue, an osteo-conductive porous surface can be formed if desired. This osteo-conductive porous surface can be ceramic-based or metal-based, and may include polymeric components, or be a combination of any of those. It can also be partially or completely bioresorbable (biodegradable), to promote bone fixation by osteo-induction or osteo-conduction. Such a porous surface can be obtained by any conventional means known to those skilled in the art, for example, but not limited to, by particle sintering, thermal spray coating, milling, etc.

Regions of the surface can also be smooth, particularly in regions where the prosthetic bone is isolated from the host environment. Such a smooth surface is preferably constituted of a biocompatible polymer, thermoplastic in nature, and may contain different types of fillers, for example, but not limited to, short, long or continuous fibers, whiskers, or particles. Whether or not composite in nature, a suitable surface can be obtained by any conventional means of surface coating, such as overmolding, wrapping, thermal spraying, electrostatic coating, etc. Alternatively, a smooth surface can be achieved by simply leaving a section of the thermoplastic composite uncovered by any further surface.

Structures may be introduced at the surface of the implant by chemical (chemical vapour deposition or CVD, electrochemical coating, etc.), physical (e.g., plasma-spray coating), or thermo-mechanical means (press-fitting, polymer infiltration of porous structure, etc.)

At the surface, a bioactive coating, such as a HA coating can be used as a layer outward of the composite. HA allows the prosthetic bone to obtain osteointegration by bone ingrowth into the implanted prosthetic bone. This HA coating may be applied by thermal spraying, which may employ any method known to those skilled in the art, such as but not limited to flame spray, plasma, cold spray and high velocity oxy-fuel (HVOF). The coating may be applied directly to the composite structure. Specific surface treatment of the composite structure may also be employed to enhance the bonding of the thermal spray coating.

A film may be used such as the one described in applicant's co-pending PCT patent application entitled “Tie Layer and Method for Coating Thermoplastics” filed on Jan. 13, 2006, the entirety of which is herein incorporated by reference. Briefly, a tie layer of a compatible composite matrix containing a filler can be used as a tie layer or film prior to application of a surface having an osteo-conductive porous region. The tie layer may be adequately compatible so as to become co-molded onto the thermoplastic composite through application of heat. The filler which may be contained in the tie layer is one having a fiber, particle or other type of particulate that maintains its structural integrity when exposed to heat capable of melting the compatible thermoplastic matrix. An exemplary tie layer may contain from 2 to 70% filler, and the remainder may be the compatible matrix. After application of such a tie layer, a surface layer may be applied, such as one containing ceramic or metallic components. Should such a surface be applied through conventional means, such as be heating or plasma application, the tie layer serves to shield the fiber-reinforced thermoplastic composite form heat deformation or destruction while the surface layer becomes bonded to the prosthetic bone.

Physical properties of the Prosthetic Bone. The nature and structure of the composite, surface and the optional polymer-based core are selected in such a way as to give the stem a good match with the physical properties of regular bone. For example, regular bone may have an elastic modulus or rigidity of between 5 GPa and 30 GPa, viscoelastic properties of, for example, a damping factor tan δ≈0.02-0.04, and a specific weight of approximately 0.4-2.1 g/cm³. The prosthetic bone may be formed so as to emulate these physical properties, or to be in a range that is near to the range expected for bone. Exemplary ranges for specific weight are from 0.2 to 4.0 g/cm³, with the ranges of 0.4 to 4.0 g/cm³, or 0.4 to 2.1 g/cm³ being acceptable.

The general structure of the prosthetic bone is advantageously similar to real bone. In particularly, as discussed herein, the interior or core of the bone may be selected so as to be less dense than the composite layer. Finally, the surface may be selected to provide appropriate hardness, strength, biocompatability, bioresorptive and osteo-conductive or osteo-inductive characteristics.

The Core. The core can have any appropriate shape and properties that allow the volume-related characteristics of the bone to be replaced to be matched when the total prosthetic bone is formed. Density, rigidity or stiffness, rigidity/weight ratio, or strength/weight ratio are such parameters that can be considered. The core may be formed of a less dense polymer, or may even be left hollow (filled with air), if it is desirable to achieve the overall stiffness and strength required to match real bone. Any material as may be known to those skilled in the art could be included in the core.

As fillers for the core, intrinsically soft polymers such as thermoplastic urethanes (TPUs), linear low density polyethylene (LLDPE), block copolymers such as SBS (styrene-butadiene-styrene), silicone rubbers, metallocene thermoplastic olefins or low-modulus polymeric foams, such as polypropylene foams, polyethylene foams, polyamide-imide foams, thermoplastic polyamide foams, polysulfone foams, may be used, but core materials are not limited to these.

Method of Forming Prosthetic Bone. In order to form a prosthetic bone according to the invention, the following steps may be used. If other combinations of steps can be employed to arrive at the same prosthetic bone as described herein, this would also fall within the scope of the invention. Methods provided herein are exemplary in nature only.

In general, the prosthetic bone can be formed by preparing the fiber-reinforced thermoplastic composite as a hollow structure, into which a core can be inserted, or by forming the thermoplastic composite around a core.

The composite is prepared having a shape emulating a bone to be replaced. In the instance where a hollow structure is formed, the composite is formed around a form, such as a bladder. An exemplary fiber structure may be a dry woven braid of carbon fiber composite, which may have long fibers of 5 mm or greater in length. An exemplary length is about 1 inch. Such a fiber composite may take on the appearance of a sock, when surrounding the form or bladder. The fiber length would be above the critical fiber length, which would be understood by a person of skill in the art. The fibers may be woven or braided into longer fibrous structures, which may ultimately be the length of the entire prosthetic bone. Heat and/or pressure may be applied to consolidate the thermoplastic composite to allow the composite to harden in the shape of the desired bone. The temperature applied is above that of the melting point of the thermoplastic. For example, in the case where CF/PA12 (carbon fiber in a PA12 matrix) is used, the mold or bladder is heated to 200-240° C., which is above the melting point of the polymer.

Because the fiber reinforces the structure, adequate strength can be achieved, allowing for variability of the stiffness or rigidity of the composite. Different thicknesses or densities of the composite layer allows for adjustments in the stiffness, density and strength of the composite layer. A number of fiber layers may be placed on the composite to achieve the desired properties.

A core may optionally be inserted within the hollow portion of the thermoplastic composite.

Subsequently, the composite is coated with an osteo-conductive material, and a porous region is formed on at leas one surface of the composite. In some instances, or for portions of the thermoplastic composite, the surface may be modified or otherwise prepared to receive a further surface coating. For example, a film may be used such as described in applicant's co-pending PCT patent application entitled “Tie Layer and Method for Coating Thermoplastics” filed on Jan. 13, 2006, the entirety of which is herein incorporated by reference.

Briefly, such a thin film formed of from 2 to 70% filler and the remainder being a polymeric matrix compatible with the fiber-reinforced thermoplastic core, and having thickness of 1 mm or less could be used to prepare the surface to receive a biocompatible layer of ceramic or metal. In the case where such a thin film is used, the film layer is wrapped around the composite and molded to take on the shape of the bone to be replaced. The thickness contributed by this layer is accounted for in the overall bone size. Once the film is melted onto the composite layer, it is cooled, and any method known to those skilled in the art is used to apply the ceramic and/or metal surface. The surface may be prepared, such as by sand-blasting or roughing up the surface so as to expose portions of the filler. Application of the surface layer via a conventional atmospheric plasma spray device may be used.

In an embodiment of this method, hydroxyapatite (HA) is a preferred biocompatible ceramic for deposition on the surface. However, other ceramics, or mixtures of ceramics combined with metals or polymers may be used (such as titanium oxide). Typically in atmospheric plasma spraying, a porosity of about 10% or greater is achieved in certain regions of the surface of the prosthetic bone. Additionally, roughness on the surface can be used to accomplish the osteo-conductive region.

The surface can be porous or rough, or may have a combination of porosity and roughness. Having a porous surface is optional. The surface generally includes a region of roughness, which may be classified as meso (100-500 μm), micro (1-50 μm) or nano (<<1 μm) roughness (or rigosity). An example of surface roughness which may be used according to an embodiment of the invention is micro roughness. A different type of roughness may be selected depending on preferences related to a certain application or material.

Slightly porous regions of the surface may be achieved so as to create an osteo-conductive regions, allowing adjacent bone tissue to grow. A region of roughness can be created so that the roughness of the surface provides a region of osteo-conductivity. A certain degree of roughness, which includes nano roughness levels may be adequate to establish osteo conductivity.

Advantageously, the invention allows formation of a prosthetic bone having strength, toughness, impact- and fatigue-resistance capable of providing a stem life expectancy that meets or surpasses the desired implantation period.

### EXAMPLES

### Example 1

### Total Hip Prosthesis (THP)

In this example, the inventive prosthetic bone according to the invention is a biomimetic THP stem.

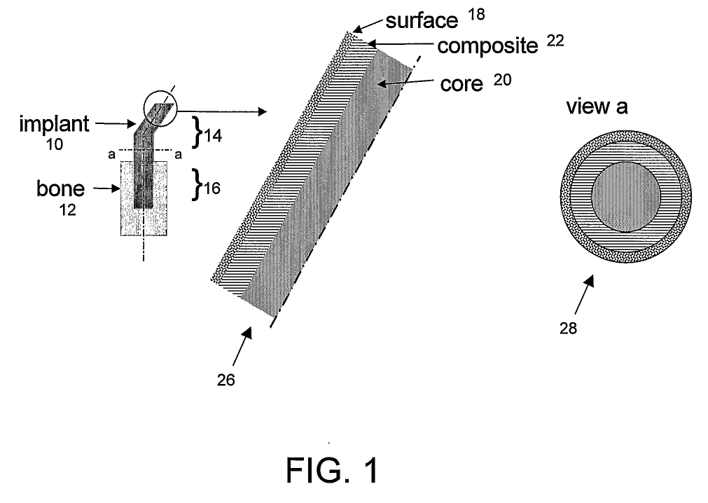

FIG. 1 illustrates a prosthetic bone according to the invention, in this case formed as a THP stem or “implant” (10) to be implanted in hip replacement surgery, to be inserted in the femoral bone (12). The stem has an extra-osseous end (14) and an intra-osseous end (16). The surface (18) of the implant is designed so that fixation of the implant to the host tissue, either by adhesive bonding or by bone integration, allows a good stress transfer between the implant (10) and the bone (12) at any point along the implant (stem). At any point along the stem, its stiffness adjacent to the bone approximately matches that of the bone, making stresses in the bone and the stem approximately equal in the vicinity of the bone-stem interface. Section a..a illustrates the 3 layers: polymer-based core (20), fiber-reinforced thermoplastic composite (22) and surface (18), illustrated in both longitudinal section (26) and cross-section (28).

The stem has a solid or hollow, cylindrical or frustoconical structure, or may take on any other shape suitable for the insertion in the portion of the femoral bone to be replaced. The fiber-reinforced thermoplastic composite is made of several concentric layers of a biocompatible polymer composite with specific fiber orientations to obtain the strength and rigidity required from the stem. In this case, the composite is a continuous fiber reinforced polymer composite. The structure may be obtained by conventional thermal consolidation (pressure and heat applied over a period of time to ensure complete melting of the polymer matrix and complete wetting of the fibers, followed by controlled cooling until complete solidification) using one of the following processes: inflatable bladder molding, compression molding, filament wounding and filament braiding.

As the surface, a bioactive hydroxyapatite (HA) coating is added as a layer outward of the composite. Hydroxyapatite allows the THP stem to obtain osteointegration by bone ingrowth into the THP stem. This HA coating may be applied by thermal spraying (such as but not limited to flame spray, plasma, cold spray and high velocity oxy-fuel) applied directly to the composite structure. Specific surface treatment of the composite structure may be necessary to ensure adhesion of thermal spray coating. The structure include an internal core with such shape and properties that the volume-related characteristics of the femoral bone can be obtained (density, rigidity/weight ratio, and strength/weight ratio). The stem permits adaptation to different commercially available femoral heads, depending on the preference of the orthopedic surgeon.

### Example 2

### Fiber-Reinforced Composite

To illustrate the bone-matching properties of the composite, a CF/PA12 composite having 68 wt % long carbon fibers and 32 wt % polyamide 12 was compression-molded in different lay-up configurations (fiber orientations) and tested for flexural and interlaminar resistance using standard testing methodology (ASTM D790/D2344). The results showed that, depending on the molding configuration, the moduli obtained ranged between 8 and 36 GPa and the mechanical strengths between 134 and 565 MPa. Thus the moduli which were obtained for a THP stem made of these composites correspond to those reported for dense bones (5-30 GPa). At the same time, the mechanical strength of these composite stems proved to be significantly above that of dense bones (100-200 MPa), showing that in extreme physiological conditions the composite stems of the invention would be subjected to stresses considerably below those leading to their failure. The latter property is advantageous because most technologies aiming at reproducing bone moduli, in particular technologies based on porous metals (such as but not limited to Ti, Ta, Ti—Ni) or foamed structures, often provide stems lacking adequate mechanical strength to ensure their reliability (ultimate strength below 100 MPa).

Preliminary fatigue testing was carried out in several mechanical conditions corresponding to real physiological loading levels for the hip, including extreme physiological loading, i.e., the peak load during a jump (10,000 N). In these mechanical conditions, no fatigue failure was noted for the CF/PA12 composite after 5,000,000 cycles. Considering that the hip experiences close to 1,000,000 cycles annually in normal conditions (load below 3,000 N) and using the above fatigue results at different loadings, a fatigue life above 20,000,000 cycles or 20 years can be expected for the composite stems of the invention, based on the Miner rule for fatigue life estimates from loading history.

### Example 3

### Bioactive HA Coating

To evaluate the feasibility of HA coatings of acceptable adhesion on the composite stems of the invention, flat coupons of CF/PA12 composite were prepared and coated by plasma spraying.

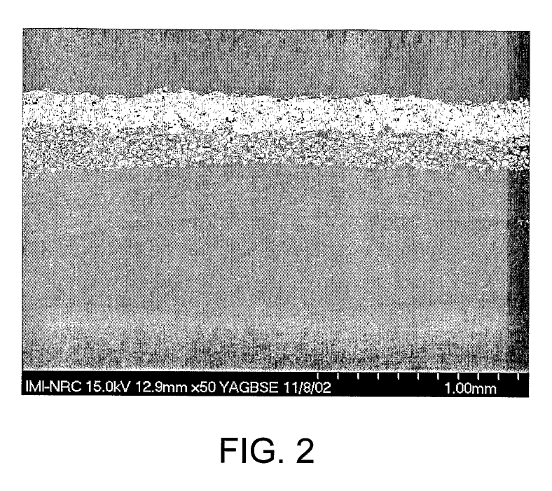

FIG. 2 illustrates an exemplary surface of HA coating on a CF/PA12 composite with a film interlayer. The film interlayer is composed of 25% vol. in HA particles (mean diameter of 30 μm) in a PA12 matrix. This layer was obtained by incorporating HA particles in a PA12 matrix using a twin screw extruder (TSE) and pelletizing the PA12/HA compound. Then a 200-300 μm-thick film was produced from the pellets of this compound using a cast film line extruder. A composition of 25% (v/v) HA/PA12 for the compound was used. The film was then overmolded on the CF/PA12 composite cylindrical structures by inflatable bladder molding in a closed mold placed into a heated press. The resulting part was then coated with HA using plasma spray.

Results showed that an HA-filled polymer film affixed to the substrate surface prior to thermal spraying led to excellent results. The HA coatings showed very good integrity and adherence values above 21 MPa based on pull tests (ASTM C633), which is considered a standard value for thermal spray coatings in an aircraft turbine engine.

Given the geometry of THPs and the physiological loads involved, the shear stresses at the surface of an implanted stem can be estimated in the 2-6 MPa range. Shear testing of the HA-coated composite coupons (ASTM D3163) showed that the shear strength of the coatings varied between 14 and 27 MPa. Preliminary shear fatigue testing of the coated composite coupons (ASTM D3166) showed that at the maximum physiological shear stress of 6-7 MPa no fatigue was observed after 5,000,000 cycles. Considering the difference between the shear stresses involved, the shear strength of the coatings and the shear fatigue life at maximum physiological shear stresses, it appears that HA coating adherence is sufficient, at least on the flat composite coated coupons, to withstand the physiological conditions of an implanted THP.

The bioactivity of these HA coatings has also been studied. The results showed that the plasma-sprayed HA coatings are highly crystalline (≧65%), with the hexagonal JCPDS Standard 9-342 for HA representing above 99% of the crystalline phase.

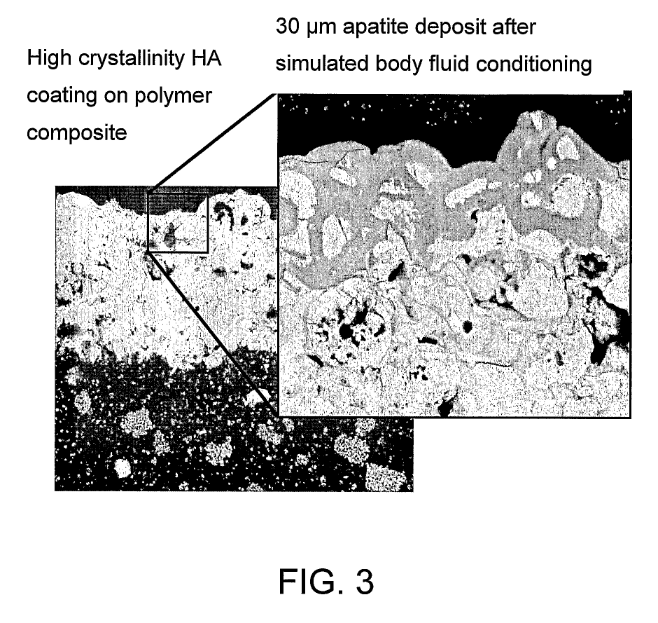

FIG. 3 illustrates a deposit of synthetic HA coating upon simulated body fluid (SBF) conditioning. A synthetic apatite deposit (upper right) or approximately 30 μm in thickness, was formed on plasma-sprayed crystalline HA coating and subjected to 28-days of SBF conditioning at 37° C.

### Example 4

### Cytotoxicity of the Materials

The biocompatibility of materials used in the prosthetic bone is an important for in vivo application. Biocompatibility was assessed on the basis of cytotoxicity testing using the MTT assay based on the work of Mosmann (Mosmann T. 1983, J. Immunol. Methods, 65: 55-63). The test consists of quantification of the survival rate of living cells (L-929 mice fibroblasts) after their exposure to extracts obtained from the tested materials. The survival rate is measured spectrophotometrically, by quantifying the capacity of living cells to transform a soluble salt into blue formazan crystals under the action of mitochondrial enzymes.

The preliminary biocompatibility results showed that the CF/PA12 composite and the bioactive HA coating formed according to Example 3, did not significantly affect the cellular viability. These results indicate that the materials used for the prosthetic bone of the present invention do not pose biocompatibility problems.

### Example 5

### Biomimetic Polymer-Composites for Prosthetic Bone

Total hip arthroplasty is subjected to long-term bone remodeling because inert synthetic materials, especially metals, involved cannot mimic the biological and biomechanical functions of bones. Important causes of this incapacity are stress shielding and migration, attributed to the difference in stiffness between cortical bone and metallic stem and lack of fixation of implant to bone. A solution to this is to develop femoral stems with bone-matching modulus and bioactive surface for osseointegration. The development of such biomimetic femoral stems based on composite materials is presented. They are composed of an internal low density core, a carbon fiber-reinforced polymer composite molded into a hollow conical-shaped stem formed by inflatable bladder compression molding, an intermediate bioactive compound and finally a plasma-sprayed bone-like hydroxyapatite coating. Results concerning the physico-chemical and mechanical characteristics of the biomimetic THP stem will be presented, including strength, bone-matching rigidity and fatigue. The manufacturing of the stem itself, including the optimal molding conditions will be described as well as preliminary testing regarding biocompatibility and finite element validation.

The object of this example is to apply this concept to a hip prosthesis by duplicating the bone structure and properties.

Table 1 shows general characteristics including density, compressive modulus and strength of different materials as well as bone.

TABLE 1
Characteristics of Bone Tissue and Prosthetic Bone Materials

| Material/ | Density | Modulus | Strength |
|---|---|---|---|
| Tissue | (g/cm³) | (GPa) | (MPa) |
| Cancellous Bone | 0.03-0.12 | 0.04-1.0 | 1.0-7.0 |
| Cortical Bone | 1.6-2.0 | 12-20 | 150 |
| Titanium | 4.4-4.7 | 106 | 780-1050 |
| Stainless Steel | 7.9 | 210 | 230-1160 |
| Ceramic (Alumina) | 3.9 | 365 | 6-55 |
| Polymer (PE) | 0.95 | 1 | 30 |

Materials and Methods. The composite fabric used to manufacture the stem is made up of a polyamide 12 (PA12) matrix with long discontinuous carbon fiber reinforcement (CF), respectively 32% and 68% in weight. In its initial state, this material comes in the form of braided non-consolidated composite tubes with a fiber orientation varying between 20 and 45 degrees. By varying the orientation of each layer of the composite stem, the properties of the multi-layer structure in different directions can be controlled.

Hip stems were manufactured by inflatable bladder compression molding, which combines compression molding, i.e., using two heated plates to simultaneously compress a given material, and bladder molding, i.e., molding a hollow tubular structure using an internal bladder. The braided tubes are mounted onto an internal inflatable bladder and then inserted in a mold cavity. The mold is closed and placed into a heated press set at a given temperature and pressure for a predetermined amount of time. Bladder is then inflated once predetermined temperature is reached.

The molding temperature varied between 175° C. and 260° C. while pressure and time were held constant (P_{eff}=50 psi, 5 min). Effective bladder inflation pressure (pressure applied minus pressure necessary to inflate bladder) was then varied between 30 and 90 psi at determined optimal temperature. A fixed molding time of 4 minutes. Finally, molding time was varied between 1 and 10 minutes at optimal pressure and temperature.

Consolidation quality was monitored by microscopic observation of polished cross-sections of the composites to verify the presence of voids or incomplete melting. Consolidation was also monitored by comparing composite density using Archimedes' method to nominal density of the composite (1.42 g/cm³).

The design of this biomimetic hip stem is based on the bone structure therefore, its structure has two principal parts that mimic two types of bone found in the femur and an external coating that promotes osseointegration.

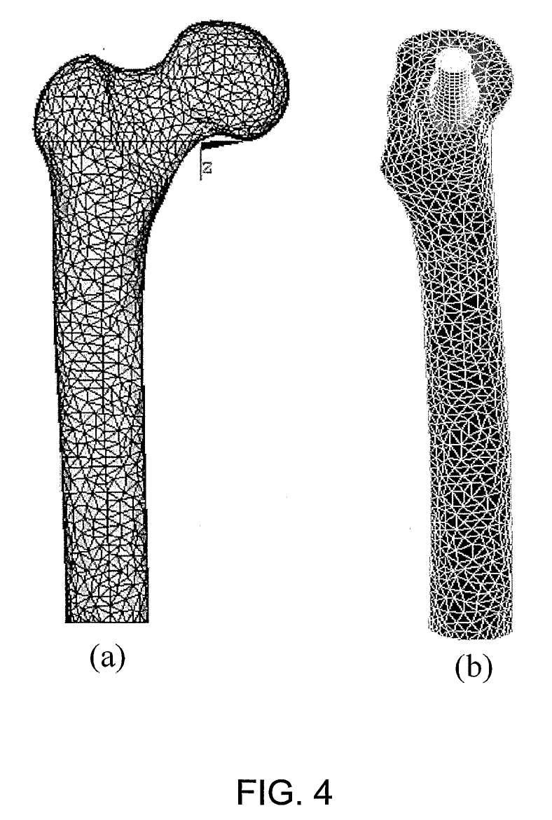

FIG. 4 shows the 3-D finite element models used to validate our design. The intact femoral bone is modeled as (a), while the femoral bone with a composite prosthesis is shown as model (b). The internal core has similar properties to those of cancellous bone. It is designed to have a low density, specific volume properties and a good adherence to the CF/PA12 sub-structure. The composite sub-structure has characteristics that resemble those of cortical bone and it was elaborated in order to improve the long-term reliability and the mechanical resistance of the stem. Precisely, the stem consists of six layers of composite braided tubes that have a particular ply configuration: the first two layers are oriented [±450], then one layer with a [0/90°] orientation and the last three have, once again a [±45°] orientation. As for the last layer, the coating, it allows the composite structure to interact properly with the host tissue. It is the element that makes this design so innovative and unique. A thin layer of bioactive compound is first laid on the entire surface of the prosthesis and then, a plasma-sprayed HA coating covers the proximal part of the stem.

Mechanical Properties. An electromechanical tester with computer data acquisition was used for the various properties that had to be tested. Uniaxial compression was done on stem samples having a specific geometry. In this case, cylindrical hollow tubes of varying lengths with a thickness of approximately 3 mm were employed.

Compression testing was done using an electromechanical testing machine with 100KN load cell with parallel plates. The crosshead speed was 5 mm/min and samples 44 mm long with parallel extremities were tested. Compressive strength and strain were calculated from the load-deflection curves at the maximum load value. Young's Modulus (E) was calculated from the following relation shown as Equation (1) where Poisson's ratio (v) is fixed to 0.3 for the CF/PA12 composite and K is the bulk compressive modulus of the material obtained from the load-deflection curve slope.

$$K = \frac{E}{3 ( 1 - 2 ( v )} \qquad ( 1 )$$

Three-dimensional models were also constructed and analyzed using FE modeling software ANSYS9.0. One model represented the intact femoral bone, while two others were made for the CF/PA12 stem and the Ti stem embedded in the femoral bone. The 3-D anatomic model of the human femur was obtained from computerized tomography (CT) scan cross-section. For structural analysis, the trabecular bone was assumed to be linear isotropic and homogeneous while the cortical bone was described as linear elastic and orthotropic. As for the Ti (E=110 GPa, v=0.3) and CF/PA12 composite stems, they both had the same geometry and were used to evaluate the performance of the developed composite compared to the traditional ones. The load case used corresponded to the most critical load case of gait (a single limb stance phase) and consisted of a 1.9 kN load applied to the femoral head and a 1.24 kN abductor load. This load was decomposed according to the anatomical plans: sagittal plan, frontal plan and transverse plan.

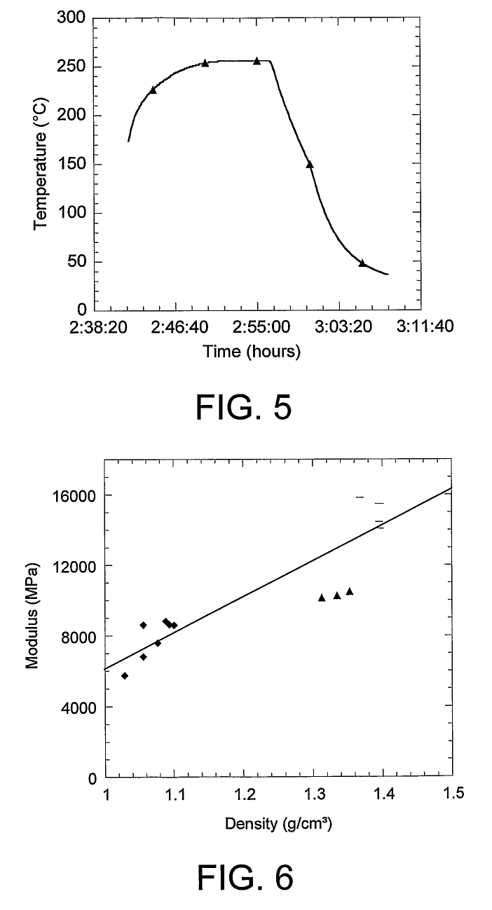

Results and Discussion. A typical molding cycle is shown in FIG. 5. The molding cycle of a hip stem is shown, indicating a first rise in temperature to 250° C., maintained for 4 minutes, while the matrix melts and fibers are wetted, then rapid cooling (17° C./min), followed by crystallization and complete solidification. FIG. 5 illustrates the different steps in a thermal molding cycle recorded under optimal conditions. Depending on consolidation conditions, different densities were obtained for the composite, varying according to the compressive modulus, as shown in FIG. 6.

FIG. 6 shows variation of modulus as a function of density depending on the consolidation quality (♦ poor: 175° C., 5 minutes, 50 psi; ▴ medium: 250° C., 4 minutes, 40 psi; [symbol] excellent: 250° C., 4 minutes, 50-90 psi).

At 175° C. maintained for 5 minutes and with an effective pressure of 50 psi, consolidation quality was poor and hence the density was extremely low, ranging between 1.0 and 1.1 g/cm³.

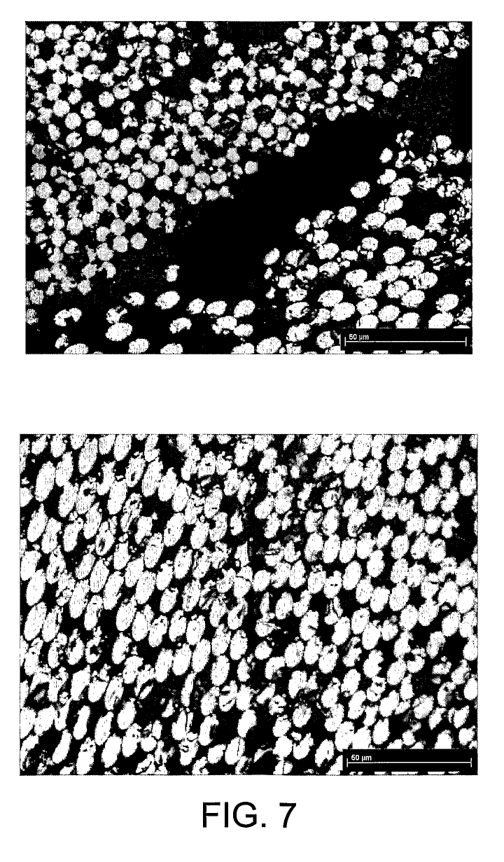

FIG. 7 illustrates this poor consolidation where high void content, as measured by apparent density, and numerous resin pockets can be observed, as seen in the upper micrograph. The two micrographs are of stem samples cut and polished in the horizontal plane with: (upper) poor consolidation quality and (lower) high consolidation quality. Resin pockets (dark grey) and large void (black) can be observed in the upper micrograph. Dark spots in carbon fibers correspond to damage created by polishing method.

These poor conditions consequently gave mediocre mechanical properties. The compressive modulus was very low, varying between 5000 and 9000 MPa, which is about half the expected value (˜16 GPa). In medium conditions, 250° C. kept for 4 minutes with an effective pressure of 40 psi, the consolidation was greatly improved because the density was significantly better (1.30-1.36 g/cm³) and closer to the density of cortical bone (1.6-2.0 g/cm³). However, having a higher density did not increase the rigidity of the stem considerably. In fact, values for the compressive modulus were all close to 9000 MPa. Finally, in the best conditions, 250° C. for 4 minutes and a pressure varying between 50 and 90 psi, the density was the closest to the theoretical value of the CF/PA12 composite (1.42 g/cm³). The consolidation was excellent and almost all void was eliminated, as shown in the lower micrograph of FIG. 7. For that matter, less than 2% void content was evaluated in these stem samples therefore, the modulus was highest and closest to that of cortical bone. This analysis on the consolidation quality and how it affects mechanical properties of the composite stems enabled us to conclude that best results and consolidation were obtained at an optimal temperature of 250° C., maintained for 4 minutes and with an effective pressure greater than 50 psi.

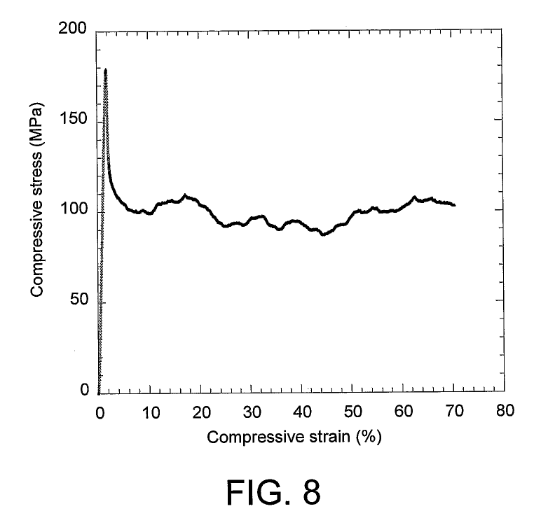

Composite stems were submitted to compression testing. FIG. 8 illustrates a typical compression stress-strain curve for a composite stem. This curve shows a typical linear elastic region ending when maximum strength is reached, where failure by buckling or barreling will occur. The slope in linear elastic region represents the compressive modulus, K. Using Eq. 1, values for the Young's modulus, E, can be calculated. Compression results are summarized in Table 2.

TABLE 2
Compression test results of stem samples obtained in optimal
molding conditions, compared to cortical bone properties

| Stem | Modulus | Strength | Strain at Maximum | Density |
|---|---|---|---|---|
| # | (GPa) | (MPa) | Strength (%) | (g/cm³) |
| 1 | 15.5 | 167 | 1.94 | 1.39 |
| 2 | 14.0 | 177 | 1.88 | 1.40 |
| 3 | 14.5 | 179 | 1.83 | 1.40 |
| 4 | 15.8 | 178 | 1.70 | 1.37 |
| 5 | 15.4 | 217 | 1.85 | 1.40 |
| Mean | 15.1 | 184 | 1.84 | 1.39 |
| StDev | 0.8 | 19 | 0.09 | 0.01 |
| Cortical Bone | 12-20 | 150 | 1-3 | 1.6-2.0 |

These results indicate that femoral composite stems have excellent mechanical properties that resemble those of the cortical bone in the human femur. In fact, femoral composite stems present physical characteristics much closer to those of cortical bones than any material presently used in the fabrication process of total hip prostheses, as shown in Table 1. Their density (˜1.40 g/cm³) is similar to that of cortical bone, which varies between 1:6 and 2.0 g/cm³ while metallic materials have densities that can be up to five or ten times greater than that of bone. Its rigidity and stiffness, respectively have an average value of 15.1 GPa and 184 MPa, while cortical bone has values of rigidity that range between 12 and 20 GPa and an ultimate strength of 150 MPa.

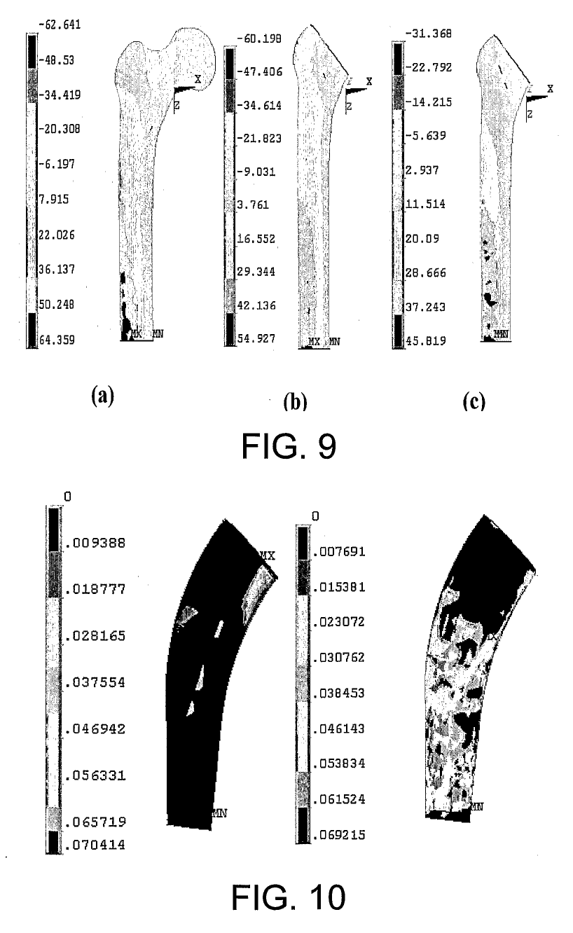

Another important aspect of this study was to validate and evaluate the feasibility of the present design with 3-D finite element analysis. The models used were shown previously in FIG. 4. Once submitted to typical physiological loading conditions, the behavior of the composite prosthesis once implanted in the femoral bone was evaluated. The same analysis was done with a titanium (Ti) implant and with an intact femur. The maximum stress distribution in the femoral bone is shown in FIG. 9. Maximum principal stress (MPa) is shown in: (a) intact femoral bone, (b) femoral bone with composite prosthesis and (c) the femoral bone with Ti prosthesis.

FIG. 9 indicates that the stresses in the surrounding femoral bone are higher when using a less rigid composite stem than a stiff Ti one. In fact, the stress distributions are similar in the intact femoral bone and in the bone with an embedded composite prosthesis. However, the stress levels in the femoral bone embedded with a Ti prosthesis are significantly lower. Since lower stress levels in bones lead to resorptive bone remodeling or bone loss, these results indicate that the Ti prosthesis will potentially lead to important bone resorption.

Also, it is important to analyze the occurrence of micromotions knowing that the incidence of migration is a predominant cause of hip implant failure. Micromotions, expressed as the contact sliding distance at the proximal bone-implant interface, are shown in FIG. 10. Contact sliding distance (migration) at the proximal bone-implant interface is illustrated for the CF/PA12 and Ti prostheses. FIG. 10 indicates that there is a significant difference between the composite and the Ti prostheses. Results show very low micromotions, ranging between 0 and 10 μm, almost over the entire proximal interface of the composite prosthesis. However, for the Ti prosthesis, contact sliding distances vary between 20 and 50 μm over the proximal surface of the prosthesis. This further suggests that bone ingrowth would be favorable in the case of the composite prosthesis than in the Ti one, since its micromotions are well below the acceptable in vivo limit of 150 μm.

Mechanical and structural reliability are only two important factors that need to be considered when designing orthopedic implants. The biocompatibility of these devices is also a predominant factor. Since our implants are coated with plasma-sprayed HA15, tests evaluating the adhesion of biological cells to this coating and cytotoxicity needed to be done. Following ISO-10993 standards for biological evaluation of medical devices, results revealed adhesion of osteoprogenitor mice cells to the HA coating and no visible cytotoxic effect from cellular viability.

Conclusion. The results of this example illustrate that high consolidation quality can be obtained when molding CF/PA12 by inflatable bladder compression molding. CF/PA12 composites can be used to mimic the cortical bone structure and its mechanical properties. Further, FE modeling suggests that bone-matching composite stems limit resorptive bone remodelling and aseptic loosening by reducing stress shielding and implant migration. These results demonstrate that a fiber reinforced polymer composite hip prosthesis according to the invention will have a high average life span, reducing the rate of revision surgery. CF/PA12 composite hip prostheses according to the invention provide an effective alternative to metallic implants.

### Example 6

### Forming a Biomimetic Polymer-Composite Hip Prosthesis

In this example, a biomimetic composite hip prosthesis (stem) was designed to obtain properties similar to those of the host bone, in particular stiffness, to allow normal loading of the surrounding femoral bone. This normal loading would reduce excessive stress shielding, known to result in bone loss, and micromotions at the prosthesis-bone interface, leading to aseptic prosthetic loosening. The design proposed is based on hydroxyapatite coated, hollow continuous carbon fiber (CF) reinforced polyamide 12 (PA12) composite sub-structure with an internal soft polymer-based core recently developed. Different composite configurations are studied to match the properties of host tissue. Nonlinear three-dimensional analysis of the hip prosthesis was carried out using a three-dimensional finite element (FE) bone model based on an anatomic model of the proximal part of a right human femoral bone obtained from computerized tomography (CT) scan cross-section. The performance of composite-based hip and titanium alloy-based (Ti-6Al-4V) stems embedded into femoral bone was compared. The effect of core stiffness and ply configuration was also analyzed. Results show that the stress in composite stem is lower than that in the Ti stem and that higher stresses in the femoral bone are generated in the composite stem generates than with a Ti stem. Micromotions in the composite stem are significantly smaller than those in Ti stem over the entire prosthesis-bone surface.

Materials and Methods. A three-dimensional anatomic model of the proximal part of a right human femoral bone was obtained from computerized tomography (CT) scan cross-section. Osteotomy of the upper end of the femoral bone was performed at the level of the greater trochanter. For structural analysis, the cortical bone was described as linear elastic and orthotropic, while the trabecular bone was assumed to be linear isotropic and homogeneous. The mechanical properties of cortical and cancellous bone are as follows. For Cortical bone, Young's Modulus(GPa): Eₓ=11.5, E_{y}=11.5, E_{z}=17.5; Shear Modulus (Gpa): G_{xy}=3.0; G_{xz}=3.5; G_{yz}=3.5; Poisson's ratio: v_{xy}=0.4; v_{xz}=0.4; v_{yz}=0.4. For Cancellous bone, Young's Modulus(GPa): E=1; Shear Modulus (Gpa):G=0.2; and Poisson's ratio: v=0.3.

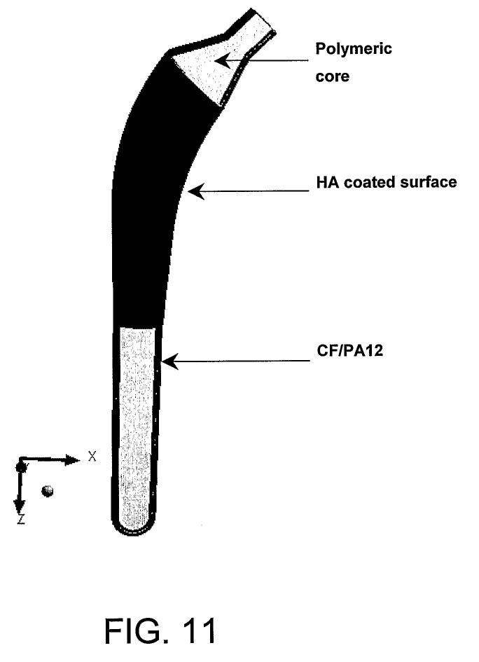

THP design. The design concept and geometry of the developed composite hip prosthesis is shown in FIG. 11. The stem includes a polymeric core, a hydroxyapatite coated surface, and comprises CF/PA12 composite. It was designed for cementless press-fit implantation to achieve initial stability. The prototype was generated using the software CATIAV5R13. It is composed of a 3-mm thick sub-structure made of several layers of a carbon fiber/polyamide 12 (CF/PA12) polymer composite laminate with pre-determined fiber orientation, an internal polymeric core and a hydroxyapatite (HA) coating in the proximal section. Optimal sub-structure thickness and different laminate fiber angle were determined experimentally by tensile and compression tests. Carbon fiber volume fraction was 55%. Preliminary biocompatibility testing showed absence of adverse cytotoxic response. Excellent bioactivity of HA coating was observed from simulated body fluid conditioning.

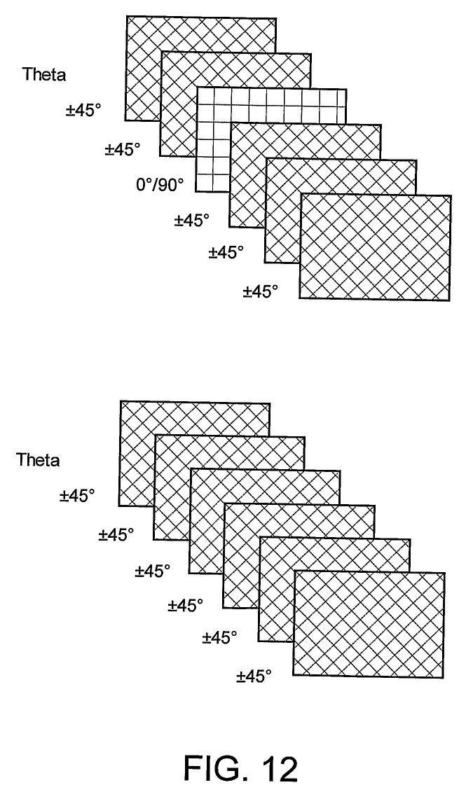

Two fiber architectures were used in the present study. Configuration I had two plies oriented at (±45), one at (0/90) and three others at (±45) ([(±45)]₂[(0/90)]₁[(±45)]₃). Configuration II had all six plies oriented at (±45) [(±45)]₆. A schematic of these two configurations is shown in FIG. 12. The composite was manufactured by inflatable bladder compression molding. The material properties assigned to the composite hip prosthesis, according to the ply configurations, are shown in Table 3.

TABLE 3
Mechanical Properties of Composite Prostheses

| | | Young's | Shear | Poisson |
|---|---|---|---|---|
| | | Modulus | Modulus | ratio |
| Prosthesis | | (GPa) | (GPa) | (ν) |
| Polymeric core | | E = 0.1, 0.4, 1 | 0.1, 0.15, 0.2 | 0.2 |
| CF/PA12 | [(±45)₂][(0/90)₁] | Eₓ = 3.5 | G_{yz} = 2.5 | 0.3 |
| composite | [(±45)₃] | E_{y} = 16.4 | G_{zx} = 3.0 | 0.3 |
| | | E_{z} = 16.4 | G_{xy} = 3.0 | 0.3 |
| | [(0/90)]₆ | Eₓ = 3.0 | G_{yz} = 2.0 | 0.3 |
| | | E_{y} = 10.7 | G_{zx} = 2.5 | 0.3 |
| | | E_{z} = 10.7 | G_{xy} = 2.5 | 0.3 |

The proximal part of the stem sub-structure was coated with 100 mm thick bioactive HA to enhance bone ingrowth and increase the fixation strength. As for the shape of the stem prosthesis, it was straight and followed the antecurvation of the shaft of the femoral bone. The composite hip prosthesis had an oval cross-section and a shaft-neck angle (CCD) of 135°.

Finite element analysis. Three dimensional models were constructed and analyzed using FE modeling software ANSYS9.0. The first model represented the intact femoral bone and the other models represented respectively Ti and CF/PA12 composite prostheses embedded in the femoral bone. The Ti stem (E=110 GPa, v=0.3) and CF/PA12 composite were used to assess the performance of the developed composite compared to the traditional ones. The same stem geometry was used for both materials.

The FE composite model was made of two types of elements: 3-D structural solid element (SOLID45-8 nodes) was used to simulate femoral bone and internal core, and a multi-layer linear structural shell element (Shell99-8 nodes) to simulate the composite sub-structure. The prosthesis-bone interface was modeled using surface-to-surface contact elements (CONTA174 and TARGE170). The complete FE model involved 22682 nodes, 114613 elements and 14782 contact elements.

Loads and boundary conditions. Most studies conducted on stress shielding have shown that the prosthesis-bone interface is an important factor for the long-term success of any implant. A frictional model was used at the prosthesis-bone interface, allowing compressive and shear stresses transfer. Two values of friction coefficient, m, were chosen: a value of 1 was assigned to the prosthesis-bone interface in the proximal, HA coated region to simulate perfect bone-HA bonding conditions and a value of 0.6 elsewhere at the prosthesis-bone interface based on experimental measurement. A fully bonded contact was assumed at the composite sub-structure-core interface.

Two load cases were considered. Load case 1, consisting of a 3 kN load applied to the femoral head with an angle 20°, was used to validate the finite element model used here by comparing stresses calculated in the Ti stem of the present model to stresses obtained by Akay (Journal of Biomedical Materials Research 1996; 31:167-182) and by Prendergast et al. (Clinical Materials 1989; 4:361-376). Load case 2 corresponded to the most critical load case of gait (a single limb stance phase) and consisted of a 1.9 kN load applied to the femoral head and a 1.24 kN abductor load. This load was decomposed according to the anatomical plans: sagittal plan, frontal plan and transverse plan. Load case 2 was used for all simulations. The data for the load magnitudes and their direction used for this simulation were selected from different previous studies. Several authors have reported that the physiological loading of the hip joint can be accurately represented by only applying joint and abducting forces, neglecting all other muscles. The loads were distributed over several nodes to avoid stress concentration. For all analyses, the displacement of all nodes at the distal end of the femoral bone was rigidly constrained.

Results and Discussion. The FE model was validated by comparing the results it produced to those obtained using their respective design of Ti alloy prosthesis. The maximum tensile and minimum compressive stresses obtained with the present model under a load of 3 kN at an angle of 20° are 79 MPa and 104 MPa, respectively. These values compare reasonably well with the maximum tensile and minimum compressive stresses obtained by Prendergast et al. (1989), are respectively of 96 MPa and 120 MPa for a similar loading case, whereas in the case of Akay (1996), slightly different stresses of respectively 100 MPa and 137 MPa were reported. Although not identical, these stress results obtained using the present model agree well within an acceptable range of variation with those obtained using other FE models with different geometries of Ti prosthesis. The differences in stresses should be attributed to the differences in stem design used in these two studies and not to the model used here.

Effect of ply configuration (CF/PA12 stiffness). The effect of ply configuration of the composite sub-structure on micromotions at the prosthesis-bone interface was evaluated. Micromotions are expressed as gap distance and sliding distance, i.e., micromotions in the normal and tangential directions with respect to the stem. For configurations I and II, micromotions and contact pressure were almost equal to zero over the entire contact surface, with a maximum located at the calcar region. Configuration I showed a minimum gap distance of −139 μm and a maximum sliding distance of 73 μm. Configuration II showed minimum gap distance and maximum sliding distance of respectively −181 μm and 96 μm. Configuration I thus showed peak micromotions of more than 30% lower than configuration II.

The influence of ply orientation on the maximum and minimum stresses in the femur and the prosthesis, as well as its influence on the interfacial total stress and pressure shows that ply configurations I and II led to similar peak maximum and peak minimum principal stress in the femoral bone. Maximum and minimum stress values in the prosthesis were also fairly close for ply configurations I and II, with values only slightly higher for ply configuration II (average 6%). At the prosthesis-bone interface, however, the influence of ply orientation on total contact stress and pressure is significant. The maximum contact pressure and maximum contact stress increased from 24 MPa and 34 MPa respectively for configuration I to 33 MPa and 46 MPa for configuration II. Configuration I, which had a modulus closer to that of cortical bone, performs slightly better than configuration II since it generates lower stresses in the prosthesis and lower interfacial pressures, as well as fewer micromotions at the prosthesis-bone interface.

Effect of prosthesis core stiffness. The effect of prosthesis core stiffness on the stresses and micromotions was also evaluated. Three different stiffnesses (100 MPa, 400 MPa and 1000 MPa) were used, corresponding to soft polymeric materials. Unlike the effect of ply configuration, core stiffness had a small influence on the stresses at the prosthesis-bone interface. A comparison of the maximum and minimum values of the total contact stress and pressure, obtained for each values of core stiffness, revealed only a difference of less than 2% between the results. A decrease of 7% in micromotions (gap distance and sliding distance) was observed when changing from a core stiffness of 100 MPa to 1000 MPa.

The distribution of the maximum and minimum principal stresses in the composite prosthesis using different core stiffness shows a uniform stress distribution along the composite prostheses. A non-significant reduction in the maximum and minimum stress in the prosthesis was observed. The peak maximum principal stress displayed at the shaft-neck junction dropped from a value of 72 MPa for a core stiffness of 100 MPa to a value 68 MPa for a core stiffness of 1000 MPa. Moreover, the peak minimum principal stress increased from a value of −96 MPa to −90 MPa for the same change in core stiffness of 100 MPa 1000 MPa, respectively.

Maximum principal stress distribution in the femoral bone was evaluated for different core stiffnesses. The maximum principal stress increases gradually from 5 MPa at the proximal part to 55 MPa at the distal part. The core stiffness had no significant effect on peak maximum stresses developed in the femoral bone. The variation of the peak maximum and minimum stresses observed in the femoral bone was less than 0.01%. It thus appears that the distribution of the maximum principal stress in the bone is similar for each core stiffness.

Comparison of Ti and CF/PA12 composite prosthesis. The Ti prosthesis led to a non uniform and higher stress distribution, whereas a uniform and lower stress distribution was observed in the case of the composite prosthesis. The maximum principal stress over the entire composite prosthesis surface varied between 0 and 20 MPa, while it ranged for Ti between 0 and 50 MPa. The minimum principal stress varied in the composite prosthesis between 4 MPa and −6 MPa, while it varied between 6 MPa and −30 MPa in the Ti prosthesis. This indicates that the mechanical load is mainly supported by the metallic prosthesis. A stress concentration was noted at the neck-shaft junction, where maximum principal stress in the Ti prosthesis reached a value of 79 MPa, compared to 68 MPa for the composite prosthesis.

The maximum principal stresses in the intact femoral bone and in the femoral bone embedded with a Ti or composite prosthesis found that the stresses in the femoral bone with a Ti prosthesis were significantly lower than those in the femoral bone with a composite prosthesis or in an intact femoral bone. The principle stress for the intact femoral bone ranged between −63 MPa and 64 MPa comparative to −60 MPa and 55 MPa for the bone with the composite prosthesis, and to −31 MPa and 46 MPa for the bone with Ti prosthesis. The composite prosthesis thus produced a maximum principal stress in the lateral side of the femoral bone that was very close to those produced in an intact femoral bone and 25% greater than that produced by the Ti prosthesis. This indicates that the femoral bone with an embedded composite prosthesis is sufficiently loaded, which favors bone apposition. Biologically speaking, bone apposition is simply the ongoing deposition of newly produced bone tissue by the osteoblast cells to continuously regenerate the bone. This biological process is described in mechanical terms by the well known Wolff's law, stating that the bone architecture is modeled by the mechanical stress to which it is subjected.

Alteration of the stress pattern (stress shielding) induced by the prosthesis leads to resorptive bone remodeling. The Ti prosthesis may provoke stress shielding and long term bone resorption, since the femoral bone is less loaded, while in presence of the composite prosthesis stress shielding and bone resorption are not expected to occur, or will occur with less detrimental effect than a typical Ti prosthesis.

Micromotions at the prosthesis-bone interface for the Ti and composite prostheses were evaluated. A significant difference between micromotions in Ti and composite prostheses was observed. Results for the composite prosthesis showed very low sliding distance over the entire proximal prosthesis-bone interface, as they generally ranged between 0 and 20 μm, with a peak micromotion of 70 μm. These micromotions are well below the limit shown by in vivo studies of 150 μm in micromotions for which dense fibrous tissues are generated at the prosthesis-bone interface. In addition, the composite prosthesis showed very small gap distance values over the entire contact surface. These values ranged between 0 and 33 μm with a peak minimum of 128 μm. On the contrary, an important part of the surface of the Ti prosthesis experienced sliding distance ranging between 0 and 50 μm, with a very high peak gap distance (negative micromotion) of 238 μm. These results indicates that micromotions, leading to aseptic loosening, are expected to be considerably more important for the Ti prosthesis than the composite prosthesis.

General discussion. The purpose of this example was to assess the effectiveness of the biomimetic composite hip prosthesis. The influence of the ply configuration and the core stiffness on the performance of the composite were analyzed. A comparative study between the composite and Ti prostheses was carried out. The result produced by FE models demonstrated that the ply configuration of the sub-structure had a strong effect on micromotions and stress at the prosthesis-bone interface. In the ply configuration II, an increase of 30% in micromotions and more than 35% in total contact stress and pressure was observed with respect to ply configuration I, as a result of the lower stiffness of the composite prosthesis in configuration II. However, the ply orientation had a small influence on the distribution of the stress in the femoral bone and the prosthesis. Core stiffness had however less effect on micromotions. Increasing core stiffness from 100 MPa to 1000 MPa reduced micromotion about 7%. Also, the variation of the core stiffness did not have a significant effect on the stress within the femoral bone.

Comparative study of composite and Ti prostheses revealed important differences in their effect on femoral bone. The composite prosthesis minimized total contact stress and pressure at the prosthesis-bone interface and this will prevent loosening of the prosthesis. The amplitude of micromotions for the composite prosthesis were acceptable and below the limit of 150 μm. Ti prosthesis on the contrary showed a very high gap distance of 238 μm, which exceeded the threshold value of micromotions. The composite prosthesis transferred more load to the femoral bone than the Ti prosthesis and this prevents stress shielding and bone resorption. A possible extension of the present work would be to consider bone remodeling after total hip replacement surgery, for the purpose of predicting the long-term response of host tissue to the insertion of the composite prosthesis.

The composite hip prosthesis illustrated that stem stresses are lower and more uniform with CF/PA12 than with Ti (less than 5 MPa at any prosthesis surface point). Further, the CF/PA12 stem produces small micromotions over the entire surface (max 70 μm in the tangential direction and maximum micromotions are 2 times lower the generally accepted limit (150 μm). Additionally, core stiffness did not appear to have as much effect on the bone stresses and micromotions but had an important effect on the stresses experienced by the composite structure. Thus, it is likely that successful bone ingrowth of the composite prosthesis will occur, due to the small amplitude of its micromotions. Furthermore, the CF/PA12 composite prosthesis limits stress shielding, and thus lowers bone resorption, since the femoral bone carries higher stresses. Finally, this prosthesis will lead to lower proximal migration, because the cancellous bone stresses are very small.

The above-described embodiments of the present invention are intended to be examples only. Alterations, modifications and variations may be effected to the particular embodiments by those of skill in the art without departing from the scope of the invention, which is defined solely by the claims appended hereto.

1. An implantable biomimetic prosthetic bone formed of a hollow fiber-reinforced thermoplastic composite having an inner and an outer surface, and an osteo-conductive region on the outer surface, wherein the hollow fiber-reinforced thermoplastic composite has a stiffness that matches stiffness of bone to be replaced.

2. The prosthetic bone of claim 28 wherein the osteo-conductive region of the surface comprises a region of porosity.

3. The prosthetic bone of claim 2 wherein the region of porosity comprises about 10% porosity.

4. The prosthetic bone of claim 28 wherein the osteo-conductive region of the surface comprises a region of roughness.

5. The prosthetic bone of claim 4 wherein the region of roughness comprises meso (100-500 μm), micro (1-50 μm) or nano (<1 μm) roughness.

6. The prosthetic bone of claim 28 wherein the surface comprising an osteo-conductive porous region is bonded to the thermoplastic composite using a tie layer comprising a compatible polymeric matrix and 2-70% filler.

7. The prosthetic bone of claim 1 wherein the surface additionally comprises an osteo-inductive porous region.

8. The prosthetic bone of claim 1 wherein the osteo-conductive porous region comprises ceramic, or a combination of ceramic with metal or polymer.

9. The prosthetic bone of claim 1 having an elastic modulus of between 5 and 30 GPa.

10. The prosthetic bone of claim 1 having a specific weight of 0.4-4.0 g/cm³.

11. The prosthetic bone of claim 1 comprising an extra-osseous section and an intra-osseous section, each section having a surface thereon; said osteo-conductive region being located on the surface of the intra-osseous section.

12. The prosthetic bone of claim 1 wherein said osteo-conductive region is bioresorbable or biodegradable.

13. The prosthetic bone of claim 1 wherein the surface additionally comprises a smooth region.

14. The prosthetic bone of claim 13 wherein the smooth region comprises a biocompatible polymer formed of thermoplastic.

15. The prosthetic bone of claim 14 wherein the biocompatible polymer comprises a composite structure including short fibers, long fibers, continuous fibers, whiskers, particles, or combinations thereof as filler therein.

16. The prosthetic bone of claim 1 wherein the composite structure comprises polymer-based oriented fibers; mineral-based fibers; metallic fibers; ceramic fibers; or polymer-based fibers with nanoreinforcement by nanoparticles, nanowhiskers, nanofibers or nanotubes.

17. The prosthetic bone of claim 1 wherein the fiber-reinforced thermoplastic composite is braided wound or filament wound around the polymer-based core.

18. The prosthetic bone of claim 1 wherein the fiber-reinforced thermoplastic composite comprises CF/PA12.

19. The prosthetic bone of claim 1 wherein the surface comprises hydroxyapatite, TiO₂ or a CaP-containing ceramic.

20. A method for forming an implantable biomimetic prosthetic bone comprising the steps of:
molding a fiber-reinforced thermoplastic composite into a hollow stem in the form of a prosthesis,
consolidating the thermoplastic composite with application of heat at a temperature higher than the melting point of the thermoplastic;
coating the thermoplastic composite with an osteo-conductive material; and
forming a region of roughness or porosity on the surface of the fiber-reinforced thermoplastic composite;
wherein the nature and structure of the molded fiber-reinforced thermoplastic composite are selected to provide a match of stiffness and specific weight with bone to be replaced.

21. The method of claim 20 wherein the step of coating the thermoplastic composite with an osteo-conductive material comprises applying to the thermoplastic composite a tie layer comprising a compatible polymeric matrix and 2 to 70% filler and subsequently applying the osteo-conductive material.

22. The method for forming a prosthetic bone according to claim 20 wherein the step of forming a region of roughness or porosity comprises particle sintering, thermal spray coating, or milling.

23. The method for forming a prosthetic bone according to claim 20 additionally comprising the step of forming a smooth region on the surface of the thermoplastic composite layer.

24. The method of claim 20, wherein the step of forming a smooth region comprises depositing a biocompatible polymer on the thermoplastic composite.

25. The method of claim 24 wherein depositing the biocompatible polymer comprises overmolding, wrapping, thermal spraying, electrostatic coating, chemical vapour deposition (CVD), electrochemical coating, plasma-spray coating, press-fitting, polymer infiltration, or combinations of these.

26. The method of claim 20 wherein the fiber-reinforced thermoplastic composite is braided or filament wound.

27. (canceled)

28. The prosthetic bone of claim 1 further comprising a polymer-based core wherein the polymer-based core is chosen to permit the prosthetic bone to match specific weight of the bone to be replaced.

29. The method of claim 20 further comprising selecting a polymer-based core for insertion into the fiber-reinforced thermoplastic composite to provide a match of stiffness and specific weight with bone to be replaced.

30. A method for forming an implantable prosthetic bone by inflatable bladder compression molding, comprising the steps of:
mounting a fiber-reinforced thermoplastic onto an internal inflatable bladder inserted in a mold cavity of a mold;
closing the mold;
placing the mold in a heat press and inflating the bladder once a predetermined temperature is reached; and
cooling and removing a hollow prosthetic bone from the mold.

31. The method of claim 30 further comprising applying a coating on an outer surface of the hollow prosthetic bone to improve an osseointegration of the hollow prosthetic bone with a bone.
